(19) United States
(12) Patent Application Publication
(10) Pub. No.: US 2008/0081054 A1
(43) Pub. Date: Apr. 3, 2008

(54) Aqueous Nail Polish Film

(75) Inventor: Philippe Ilekti, Maisons Alfort (FR)

Correspondence Address: OLIFF & BERRIDGE, PLC, P.O. BOX 320850, ALEXANDRIA, VA, 22320-4850, US

(73) Assignee: L'OREAL, Paris (FR)

(21) Appl. No.: 11/579,275

(22) PCT Filed: May 17, 2005
(86) PCT No.: PCT/FR05/50337

§ 371 Date: Jan. 17, 2007

Related U.S. Application Data
(60) Provisional application No. 60/583,485, filed on Jun. 29, 2004.

(30) Foreign Application Priority Data
May 19, 2004 (FR) 0451006

(51) Int. Cl. A61K 8/02; A61K 8/72; B32B 37/12

(52) U.S. Cl. 424/401; 156/62.2; 424/61

(57) ABSTRACT

The present invention relates to a flexible article for making up and/or caring for the nails and/or false nails, comprising at least:

an adhesive layer; and

at least one polymeric film obtained by evaporating the aqueous phase from an aqueous dispersion of particles of at least one film-forming polymer.

The present invention relates to a flexible article for application to false nails or nails to make them up and/or care for them.

Conventionally, nails or false nails are made up using liquid makeup compositions, still generally known as nail polish. Said nail polish is generally applied to the surface of the nail to be made up in the form of superposed layers, allowing an intermediate drying step between each application of nail polish. In reality, such a makeup method is not entirely satisfactory.

Firstly, applying it requires a certain amount of time. Further, that type of makeup needs to be re-applied within a short period because it does not stay on sufficiently well. In three to five days in general, the polish which has been applied flakes and loses its gloss. It then has to be removed and fresh makeup needs to be re-applied.

Further, conventional nail polish formulations generally involve the use of volatile solvents which generate a disagreeable odor during application.

A number of alternatives have been proposed to attempt to at least partially overcome the disadvantages mentioned above. Thus, nail makeup products in the form of a kit of two liquid nail polish compositions have been proposed. However, the improvement in staying power is achieved in that case to the detriment of the application conditions, since the number of layers to be applied is multiplied by two.

A further alternative consists of developing nail polish compositions based on a dispersion of polymers in an aqueous phase, which is thus satisfactory as regards olfactive properties. Unfortunately, the corresponding polishes turn out not to have sufficient staying power.

The aim of the present invention is to propose a method of making up and/or caring for nails or false nails which, in contrast to conventional liquid type nail polish formulations, is easy and rapid for the user to apply, stays on significantly longer, and has a significantly reduced content of organic solvent(s).

Thus, the present invention proposes an article for making up and/or caring for the nails or false nails which is in the form of an adhesive film produced from a dispersion of particles of at least one film-forming polymer in an aqueous phase.

Thus, in a first aspect, the present invention provides a flexible article for making up and/or caring for the nails and/or false nails, comprising at least:

an adhesive layer; and

at least one polymeric film obtained by evaporating the aqueous phase from an aqueous dispersion of particles of at least one film-forming polymer.

In a variation of the invention, the article further comprises at least one film of colored polish between the adhesive layer and the polymeric film.

Said film of polish is obtained by cross-linking or evaporating the organic or aqueous solvent phase from a solution or dispersion of at least one film-forming polymer.

In a variation of the invention, the polymeric film is transparent.

As used here, the term “transparent” means that the coating has a HAZEBYK index of less than 5 as measured with a KYKHAZEGLOSS type gloss meter.

In a second aspect, the present invention provides a method of preparing a flexible article for making up and/or caring for the nails, comprising at least the following steps consisting in superposing, on a removable support:

a) at least one layer of a composition based on at least one adhesive material; and

b) at least one layer of an aqueous dispersion of particles of at least one film-forming polymer, evaporation of the aqueous phase being carried out consecutively to deposition thereof to obtain a polymeric film.

In a first variation of the invention, the method comprises at least the steps consisting in:

a) depositing at least one layer of an adhesive material on a removable support;

b) depositing at least one layer of an aqueous dispersion of particles of at least one film-forming polymer on said adhesive layer; and

c) evaporating said dispersion to obtain a polymeric film.

In a second variation of the invention, the method comprises at least the steps consisting in:

a) depositing at least one layer of an aqueous dispersion of particles of at least one film-forming polymer on a removable support;

b) evaporating said dispersion to obtain a polymeric film;

c) depositing at least one layer of an adhesive material on said film obtained in b);

d) if necessary, at least partially drying the article obtained;

e) covering the adhesive layer obtained in c) by a removable support; and

f) recovering said article by peeling the polymeric film from the support in a).

In these two variations, the evaporation step may advantageously be modified to obtain a condition for said polymeric film which is only partially dry.

In a further variation of the invention, said method further comprises a step consisting in forming a film of colored polish between the adhesive layer and the polymeric film.

In a third aspect, the present invention provides a product for making up and/or caring for the nails and/or false nails comprising, in a packaging which is substantially airtight, at least one article in accordance with the invention, the packaging being such that the article is preserved in a partially dry form.

Within the context of the present invention, the term “partially dry” is intended to mean that the article obtained after forming the cross-linked film is not entirely free of residual solvent. In particular, it has a dry matter content of less than 80%, in particular less than 75% and more particularly less than 70% by weight relative to its total weight.

In a particular embodiment, said packaging comprises a reservoir, such as a pouch, which may or may not be flexible, which can contain a product in an airtight manner, to preserve said article from prematurely completely drying out before it is used.

In a fourth aspect, the present invention provides a method of preparing a product as defined above, comprising the following steps consisting in superposing, on a removable support:

at least one layer of a composition based on at least one adhesive material;

at least one layer of an aqueous dispersion of particles of at least one film-forming polymer, the aqueous phase being evaporated consecutive to depositing said composition and controlled to obtain a partially dry article; and

packaging said article in a partially dry condition within a substantially airtight packaging.

In this implementation, the article only becomes completely dry, and thus only achieves its definitive form, after application to the nail, simply by exposure to ambient air.

In a fifth aspect, the present invention provides a method of making up and/or caring for the nails using an article as defined above, comprising applying the adhesive face of the article to a natural or synthetic nail and optionally exerting pressure on the article to adhere it to the nail.

The inventors have shown that it is possible to obtain flexible articles for making up and/or caring for the nails which satisfy the requirements listed above, provided that said article has a film of polish obtained by evaporating an aqueous dispersion of particles of at least one film-forming polymer.

The article of the invention advantageously has good staying power, in particular of at least five days. It is also resistant to water, wear, and shock, and it neither wears nor flakes. Further, the article of the invention is particularly satisfactory as regards application.

Within the context of the present invention, the term “flexible” means sufficient flexibility for the article of the invention. More precisely, that article is in the form of a film which can accommodate stretch-type mechanical deformations to adjust it to the surface of a nail. This deformability is especially characterized by the deformation at break parameter, εᵣ, discussed below. In this regard, the article of the invention is distinguished from a false nail which is characterized by a stiffness that is incompatible with such mechanical deformation.

A further difference between the article of the invention and a false nail lies in the fact that this article is sensitive to polar organic solvents of the acetone, ester and/or lower alcohol type. The polymeric film on the outer face of the article of the invention, i.e. which does not adhere to the nail, can swell, which results in an increase in its weight when it is brought into contact with one of said solvents. A false nail is completely free of such sensitivity. This ability of the article of the invention to swell is advantageous since it can be eliminated when it is applied to the surface of a nail or a false nail. The article of the invention can readily be removed simply by using a conventional remover, as opposed to a false nail which has to be taken off.

### ARTICLE IN ACCORDANCE WITH THE INVENTION

The article of the invention may be characterized by a high dry extract. In the dry state, the quantity of dry material is more than 80%, in particular more than 85%, more particularly more than 90% by weight relative to its total weight. In other words, the quantity of volatile solvent is less than 20%, in particular less than 15%, and more particularly less than 10% by weight relative to the total article weight.

However, in a further advantageous embodiment, the article of the invention may advantageously be in the partially dry form. In this particular form, the article is advantageously packaged in reservoir-type packaging such as a pouch, for example, which may optionally be flexible, and which is sufficiently airtight to preserve this partially dry aspect. Advantageously, said packaging is impermeable to air and/or solvents. Only when used, and as a result when it is brought into contact with air, will the article dry completely to acquire the dry matter content defined above.

In one product in accordance with the invention, the article of the invention advantageously has a dry matter content of less than 80%, in particular less than 75%, more particularly less than 70% relative to its total weight. Said article may also have a dry matter content of more than 60%, especially more than 65% by weight relative to the total weight. As mentioned above, the partially dry article, when removed from the packaging, becomes dry as defined above within 24 hours following exposure to ambient air.

Preferably, the quantity of dry matter, usually termed the “dry extract” of the articles of the invention, is measured by heating a sample using infrared radiation with a wavelength of 2 μm [micrometers] to 3.5 μm. Substances contained in said articles and having a high vapor pressure, evaporate off under the effect of this radiation. Measuring the loss of mass of the sample allows the “dry extract” of the film to be determined. Said measurements are made using a commercial LP16 infrared dessicator from Mettler. That technique is fully described in the documentation furnished by Mettler accompanying the apparatus.

The following measurement protocol is followed.

About 10 g [grams] of sample is placed in a metal cup. After introducing into a dessicator, it is subjected to a temperature of 120° C. for one hour. The moist mass of the sample, corresponding to the initial mass, and the dry mass of the sample, corresponding to the mass after exposure to radiation, are measured using a precision balance.

The dry matter content is calculated as follows:

Dry extract=100×(dry mass/moist mass).

### Water Take-Up

The article of the invention is characterized in the dry state by a water take-up at 25° C. of 20% or less, in particular 16% or less, and more particularly less than 10%.

In the present application, the term “water take-up” denotes the percentage of water absorbed by the article after immersion in water for 60 min [minutes] at 25° C. (ambient temperature). The water take-up is measured using pieces of about 1 cm² [square centimeters] cut from the dry article. They are weighed (measurement of mass M1), then immersed in water for 60 min; after immersion, the piece of film is wiped to eliminate excess surface water then weighed (measurement of mass M2). The difference, M2−M1, corresponds to the quantity of water absorbed by the article.

The water take-up is equal to [(M2−M1)/M1]×100 and is expressed as the percentage by weight relative to-the weight of the article.

### Storage Modulus E′

Further, the article of the invention is advantageously a film having a storage modulus E′ of 1 MPa [megapascals] or more, in particular 1 MPa to 5000 MPa, more particularly 5 MPa or more, in particular 5 to 1000 MPa and still more particularly 10 MPa or more, for example 10 MPa to 500 MPa at a temperature of 30° C. and a frequency of 0.1 Hz [Hertz].

The storage modulus is measured by DMTA (dynamic and mechanical temperature analysis).

The viscoelastic tests are carried out with a DMTA apparatus from Polymer TA Instruments (model DMA2980) on a sample of the article. Specimens are cut out (for example using a punch). They have a typical thickness of about 150 μm, a width of 5 mm [millimeters] to 10 mm and a useful length of about 10 mm to 15 mm.

The measurements are carried out at a constant temperature of 30° C.

The sample is placed under tension and subjected to small deformations (for example a sinusoidal displacement of ±8 μm) during a frequency scan, the frequency being from 0.1 Hz to 20 Hz. Thus, the working region is linear, with small deformations.

Said measurements allow the complex modulus E*=E′+iE″ of the test composition film to be determined, E′ being the storage modulus and E″ the “lossy” modulus.

### Deformation and Energy at Break

Advantageously, the articles of the invention have a deformation at break εᵣ of 5% or more, in particular 5% to 500%, more preferably 15% or more, especially 15% to 400%, and/or an energy at break per unit volume Wᵣ of 0.2 J/cm³ [joules/cubic centimeter] or more, in particular 0.2 J/cm³ to 100 J/cm³, preferably more than 1 J/cm³, in particular 1 J/cm³ to 50 J/cm³.

The deformation at break and the energy at break per unit volume are determined by tensile tests carried out on an article about 200 μm thick.

To carry out these tests, the article is cut into dumb-bell shaped test specimens with a useful length of 33±1 mm and a useful width of 6 mm. The section (S) of the specimen is thus defined as: S=width×thickness (cm²); this section is used for the stress calculation.

The tests are carried out, for example, using a commercial tensile test apparatus sold under the trade name Lloyd® LR5K. The measurements are carried out at ambient temperature (20° C.).

The specimens are stretched at a displacement rate of 33 mm/min [millimeters per minute], corresponding to an extension rate of 100% per minute.

Thus, a displacement rate is imposed and the extension ΔL of the specimen and the force F necessary to impose said extension are measured simultaneously. These data, ΔL and F, are used to determine the stress a and deformation ε parameters.

A stress curve of σ=(F/S) is obtained as a function of the deformation ε=(ΔL/L₀)×100, the test being carried out until the sample breaks, Lo being the initial length of the sample.

The deformation at break εᵣ is the maximum deformation of the sample before the break point (as a %).

The energy at break per unit volume, Wᵣ in J/cm², is defined as the area beneath the stress/deformation curve, i.e.:

$${W}_{r} = {\int }_{0}^{{ɛ}_{r}} σ \cdot ɛ \cdot d ɛ$$

### Aqueous Dispersion of Particles of Film-Forming Polymers

As indicated above, the article of the invention comprises a film obtained by evaporating at least one aqueous dispersion of particles of at least one film-forming polymer. Said solid particles may be anionic, cationic, or neutral in nature.

Within the context of the present invention, the term “aqueous” means a liquid medium based on water and/or hydrophilic solvents. Said aqueous liquid medium may be essentially constituted by water. It may also comprise a mixture of water and organic solvent(s) which are miscible with water (more than 50% by weight miscible with water at 25° C.), such as ethanol, isopropanol, glycols containing 2 to 8 carbon atoms such as propylene glycol, ethylene glycol, 1,3-butylene glycol, dipropylene glycol, C₃-C₄ ketones or C₂-C₄ aldehydes.

In the present application, the term “film-forming polymer” means a polymer which can, of itself or in the presence of an auxiliary film-forming agent, form a continuous film on a support at a temperature of 20° C. to 150° C.

In accordance with the present invention, the film-forming polymer is present in the form of particles in aqueous dispersion, generally known as a latex or pseudolatex. Techniques for preparing such dispersions are well known to the skilled person.

A dispersion which is suitable within the context of the present invention may comprise one or more types of particles, said particles possibly varying in their size, structure and/or chemical nature.

The size of the polymer particles in aqueous dispersion may be from 5 nm [nanometers] to 500 nm, in particular from 10 nm to 150 nm. However, it is possible to use particles with a size of up to 1 μm.

Particle size may, for example, be measured with a Brookhaven BI-90 type apparatus using the light diffusion technique or with a Malvern Mastersizer 2000 granulometer, or by electron microscopy.

In general, the aqueous dispersion used to produce an article of the invention comprises 0.5% to 60% by weight, in particular 1% to 50% of the total dry weight of the matter in the film-forming polymer relative to the total weight of the dispersion.

In a particular implementation, the article of the invention is a multi-layered film produced in a plurality of steps from different aqueous dispersions of film-forming polymer.

More precisely, it may be a multi-layered film produced by superposing at least two or more layers respectively obtained by evaporating the aqueous phase from dispersions of particles of film-forming polymer(s) of different natures. In such a particular implementation, it is possible to use a first aqueous dispersion of at least one film-forming polymer having at least one, especially one, glass transition temperature Tg1 of 20° C. or less, especially from −120° C. to 20° C., in particular less than 10° C., especially from −120° C. to 0° C., and more particularly from −70° C. to −30° C. The dispersion is deposited on a support then dried at a temperature of 50° C. to 150° C. A second aqueous dispersion is then used, which is deposited on said first layer, said second aqueous dispersion of at least one film-forming polymer having at least one glass transition temperature, Tg2, of 30° C. or more, especially from 30° C. to 200° C., advantageously 50° C. or more, especially from 50° C. to 200° C., in particular 80° C. or more, especially from 80° C. to 180° C. The ensemble is then partially dried at a temperature of 50° C. to 150° C., in particular at a temperature of more than 100° C.

Film-forming polymers that can be used in the composition of the present invention and which may be mentioned include: synthetic polymers, of the radical or polycondensate type; polymers of natural origin; and mixtures thereof. In general, these polymers may be random polymers, block copolymers of the A-B, multi-block A-B-A or ABCD, etc type, or graft polymers.

### Radical Type Film-Forming Polymer

The term “radical type polymer” means a polymer obtained by polymerizing unsaturated monomers, in particular with ethylenically unsaturated bonds, each monomer being capable of self-polymerizing (in contrast to polycondensates).

The radical type film-forming polymers may in particular be homopolymers or copolymers, acrylic and/or vinyl.

Vinyl film-forming polymers may result from polymerizing monomers with an ethylenically unsaturated bond containing at least one acid group and/or esters of said acid monomers and/or amides of said acid monomers.

Monomers with an ethylenically unsaturated bond containing at least one acid group or a monomer carrying an acid group which may be used include a, β-ethylenically unsaturated carboxylic acids such as acrylic acid, methacrylic acid, crotonic acid, maleic acid, and itaconic acid. In particular, (meth)acrylic acid, and crotonic acid may be used, more particularly (meth)acrylic acid.

The esters of acid monomers are advantageously selected from esters of (meth)acrylic acid (also known as (meth)acrylates), especially alkyl (meth)acrylates, in particular of C₁-C₂₀ alkyl, more particularly C₁-C₈, aryl (meth)acrylates, in particular of C₆-C₁₀ aryl, and hydroxyalkyl (meth)acrylates, in particular C₂-C₆ hydroxyalkyl.

Alkyl (meth)acrylates which may be mentioned include methyl methacrylate, ethyl methacrylate, butyl methacrylate, isobutyl methacrylate, 2-ethylhexyl methacrylate, and lauryl methacrylate.

Hydroxyalkyl (meth)acrylates which may be mentioned include hydroxyethyl acrylate, 2-hydroxypropyl acrylate, hydroxyethyl methacrylate and 2-hydroxypropyl methacrylate.

Aryl (meth)acrylates which may be mentioned include benzyl acrylate and phenyl acrylate.

Particular (meth)acrylic acid esters are alkyl (meth)acrylates.

In accordance with the present invention, the alkyl group of the esters may be either fluorinated, or perfluorinated, i.e. some or all of the hydrogen atoms of the alkyl group are substituted by fluorine atoms.

Examples of amides of acid monomers which may be mentioned are (meth)acrylamides, especially N-alkyl (meth)acrylamides, in particular C₂-C₁₂ alkyl (meth)acrylamides. N-alkyl (meth)acrylamides which may be mentioned include N-ethyl acrylamide, N-t-butyl acrylamide and N-t-octyl acrylamide.

The vinyl film-forming polymers may also result from homopolymerizing or copolymerizing monomers selected from vinyl esters and styrene monomers. In particular, said monomers may be polymerized with acid monomers and/or their esters and/or their amides, such as those mentioned above.

Examples of vinyl esters which may be mentioned are vinyl acetate, vinyl neodecanoate, vinyl pivalate, vinyl benzoate, and vinyl t-butyl benzoate.

An example of a styrene monomer which may be mentioned is alpha-methyl styrene.

The list of monomers given above is not limiting and it is possible to use any monomer which is known to the skilled person which falls into the categories of acrylic and vinyl monomers (including monomers modified by a silicone chain).

It is also possible to use silicone acrylic polymers as the vinyl polymer.

Polymers resulting from radical type polymerization of one or more radical monomers may also be mentioned within and/or partially on the surface of pre-existing particles of at least one polymer selected from the group constituted by polyurethanes, polyureas, polyesters, polyesteramides and/or alkyds. Said polymers are generally termed “hybrid polymers”.

### Polycondensate

Polycondensate type film-forming polymers which may be mentioned are anionic, cationic, non ionic or amphoteric polyurethanes, polyurethane-acrylics, polyurethane-polyvinylpyrrolidones, polyester-polyurethanes, polyether-polyurethanes, polyureas, polyurea/polyurethanes, silicone-containing polyurethanes and mixtures thereof.

The film-forming polyurethane may, for example, be a polyurethane, polyurea/urethane or polyurea copolymer, which may be aliphatic, cycloaliphatic or aromatic, comprising at least one sequence selected from the following, used alone or as a mixture:

a sequence of aliphatic and/or cycloaliphatic polyester and/or aromatic origin; and/or

a silicone sequence, which may or may not be branched, for example polydimethylsiloxane or polymethylphenylsiloxane; and/or

a sequence comprising fluorinated groups.

The film-forming polyurethanes as defined in the invention may also be obtained from polyesters, which may or may not be branched, or from alkyds comprising mobile hydrogens which are modified by reaction with a diisocyanate and a bifunctional organic compound (for example dihydro, diamino or hydroxyamino), comprising in addition either a carboxylic acid or carboxylate group, or a sulfonic acid or sulfonate group, or a neutralizable tertiary amine group or a quaternary ammonium group.

Film-forming polycondensates which may also be mentioned include polyesters, polyester amides, fatty chain polyesters, polyamides, and epoxyester resins.

The polyesters may be obtained in known manner by polycondensation of dicarboxylic acids with polyols, in particular diols.

The dicarboxylic acid may be aliphatic, alicyclic or aromatic. Examples of such acids which may be mentioned are as follows: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norboranedicarboxylic acid, diglycolic acid, thiodipropionic acid, 2,5-naphthalenedicarboxylic acid, and 2,6-naphthalenedicarboxylic acid. Said dicarboxylic acids may be used alone or in combination with at least two dicarboxylic acid monomers. Said monomers particularly include phthalic acid, isophthalic acid and terephthalic acid.

The diol may be selected from aliphatic, alicyclic and aromatic diols. In particular, a diol is used which is selected from: ethylene glycol, diethylene glycol, triethylene glycol, 1,3-propanediol, cyclohexane dimethanol, and 4-butanediol. Other polyols which may be used are glycerol, pentaerythritol, sorbitol and trimethylol propane.

The polyester amides may be obtained in a manner which is analogous to that used for polyesters, by polycondensation of dibasic acids with diamines or aminoalcohols. Diamines which may be used include ethylenediamine, hexamethylnediamine and meta- or para-phenylenediamine. An aminoalcohol which may be used is monoethanolamine.

### Polymer of Natural Origin

In the present invention, polymers of natural origin may be used, which may be modified, such as shellac resin, sandarac gum, dammars, elemis, copals, cellulose polymers which are insoluble in water such as nitrocellulose, modified cellulose esters including carboxyalkyl cellulose esters, such as those described in United States patent application US 2003/185774, and mixtures thereof.

In a first variation of the invention, the aqueous dispersion of polymer particles is an aqueous dispersion of polyester-polyurethane particles and/or polyether-polyurethane particles, in particular anionic.

The anionic nature of the polyester-polyurethanes and polyether-polyurethanes used in the invention is due to the presence in their constituent motifs of groups with a carboxylic acid or sulfonic acid function.

The polyester-polyurethane or polyether-polyurethane particles used in the invention are generally sold in the form of aqueous dispersions.

Advantageously, the particle size is from 5 to 500 nm, in particular 10 nm to 250 nm.

The amount of particles in said dispersions which are currently available on the market is from about 20% to about 50% by weight relative to the total dispersion weight.

Particular anionic polyester-polyurethane dispersions which may be used in the aqueous polishes of the invention which may be mentioned include those sold under the trade names “Sancure 2060®” and “Sancure 815®” by SANNCOR.

Particular anionic polyether-polyurethanes which may be used in the invention include those sold under the trade name “Sancure 878®” by SANNCOR and under the trade name “Néorez R 970®” by ICI.

In a particular implementation of the invention, a mixture of commercially available dispersions constituted by particles of anionic polyester-polyurethane as defined above and particles of anionic polyether-polyurethane, also as defined above, may be used.

As an example, it is possible to use a mixture constituted by the dispersion sold under the trade name “Sancure 2060®” and that sold under the trade name “Sancure 861®” or a mixture of that sold under the trade name “Sancure 815®” and that sold under the trade name “Sancure 878®”, these dispersions being sold by SANNCOR.

In particular, mixtures respectively containing 60% and 70% of polyester-polyurethane particles are used, the remainder being constituted by polyether-polyurethane particles.

In this first particular variation of the invention, the dispersion of particles is used in the form of a dispersion the polyester-polyurethane and/or polyether-polyurethane particle content of which is generally from 3% to 50%, in particular 10% to 50% by weight relative to the total dispersion weight.

In a second variation of the invention, the aqueous dispersion of polymer particles is an aqueous dispersion of an acrylic polymer.

Said polymer has solubility properties at 25° C. in organic solvents corresponding to the HANSEN solubility parameters dD, dP and dH satisfying the following conditions:

dD=17.5;

dP=7;

dH=7.6;

with a radius R of 5 to 10, in particular 5 to 6.

The definition of solvents in the Hansen three dimensional solubility space is described in the article by C. M. HANSEN: “The three-dimensional solubility parameters” J. Paint Technol. 39, 105 (1967).

dD characterizes the LONDON dispersion forces derived from the formation of dipoles induced during molecular shocks;

dP characterizes the DEBYE interaction forces between permanent dipoles and the KEESOM interaction forces between induced dipoles and permanent dipoles;

dH characterizes the specific interaction forces (hydrogen bond, acid/base, donor/acceptor type, etc);

the parameters dD, dP, dH are expressed in (J/cm³)^{1/2}.

The radius R corresponds to the distance in the Hansen solubility parameter space separating an organic solvent from the point in said space corresponding to dD=17.5; dP=7; dH=7.6, with R satisfying the following relationship:

5 J^{1/2} cm ^{=3/2}≦R≦10 J^{1/2} cm^{-3/2},

in which:

R=\sqrt{4(δ^{s}_{d}−17.5)²+(δ^{s}ₚ−7)²+(δ^{s}ₕ−7.6)²}

and in which δ^{s}_{d}, δ^{s}ₚ, δ^{b}ₕ are the Hansen solubility parameters for an organic solvent for which the acrylic polymer used in the present invention has solubility properties. The definition of the radius R is known from the work by Allan F. M. Barton, CRC Handbook of solubility parameters and other cohesion parameters, Second edition, 1991, pages 95 to 109.

The acrylic polymer may be a styrene/acrylate copolymer, especially a polymer selected from copolymers derived from polymerizing at least one styrene monomer and at least one C₁-C₁₈ alkyl (meth)acrylate.

Styrene monomers which may be used in the invention which may be mentioned, for example, are styrene or alpha-methylstyrene, in particular styrene.

In particular, the C₁-C₁₈ alkyl (meth)acrylate monomer is a C₁-C₁₂ alkyl (meth)acrylate and more particularly a C₁-C₁₀ alkyl (meth)acrylate. The C₁-C₁₈ alkyl (meth)acrylate monomer may be selected from methyl acrylate, methyl methacrylate, ethyl acrylate, propyl acrylate, butyl acrylate, butyl methacrylate, hexyl acrylate, octyl acrylate, 2-ethyl hexyl acrylate, lauryl (meth)acrylate and stearyl (meth)acrylate.

An acrylic polymer in aqueous dispersion which may be used in accordance with the invention is the styrene/acrylate copolymer sold under the trade name “Joncryl SCX-8211®” by JOHNSON.

In a particular implementation of the invention, the dispersion may comprise the acrylic polymer defined above as the only polymer in aqueous dispersion.

In this second particular variation of the invention, the dry extract content of particles of polymer acrylic is such that it is effective in forming a film, especially from 3% to 60% by weight, and in particular 5% to 50% by weight relative to the total dispersion weight.

In a third variation of the invention, the aqueous dispersion used comprises a mixture of at least two film-forming polymers in the form of solid particles distinguished by their respective Tgs.

More precisely, it comprises, in an acceptable aqueous medium:

a) solid particles dispersed in the aqueous medium of a first film-forming polymer having at least one glass transition temperature, Tg1, of 30° C. or more; and

b) solid particles dispersed in the aqueous medium of a second film-forming polymer having at least a glass transition temperature, Tg2, of 0° C. or less;

the first film-forming polymer being present in an amount of 50% to 90% by weight relative to the total weight of the first and second film-forming polymers.

This dispersion generally results from mixing the two aqueous dispersions of film-forming polymer.

The first film-forming polymer has at least one, especially one, glass transition temperature Tg1 of 30° C. or more, especially 30° C. to 200° C., and advantageously 50° C. or more, especially 50° C. to 200° C., and in particular 80° C. or more, especially 80° C. to 180° C.

The second film-forming polymer has at least one, especially one, glass transition temperature Tg2 of 0° C. or less, especially −120° C. to 0° C., and in particular less than −10° C., especially −120° C. to −10° C., more particularly −30° C. to −70° C.

The glass transition temperature (Tg) of a polymer is measured by DMTA (dynamical and mechanical temperature analysis) as described below.

To measure the glass transition temperature (Tg) of a polymer, viscoelastimetry tests are carried out on a film sample using a DMTA apparatus from “Polymer Laboratories”. This film is prepared by pouring the aqueous dispersion of film-forming polymer into a Teflon matrix then drying at 120° C. for 24 hours. A film is then obtained from which specimens (for example using a punch) are cut. These typically have a thickness of about 150 μm, a width of 5 mm to 10 mm and a useful length of about 10 mm to 15 mm. This sample is then placed under tension. The sample is subjected to a static force of 0.01 N superposed on which is a sinusoidal displacement of ±8 μm at a frequency of 1 Hz. Thus, the working region is linear, with small deformations. This tensile stress is applied to the sample at temperatures of −150° C. to +200° C. with a temperature variation of 3° C. per minute.

The complex modulus E*=E′+iE″ of the test polymer is then measured as a function of temperature.

These measurements are used to deduce the dynamic moduli E′, E″ and the damping: TANδ=E″/E′.

Next, the graph of the values of TANδ is traced as a function of temperature; this graph has at least one peak. The glass transition temperature Tg of the polymer corresponds to the temperature at which the top of said peak is located.

When the curve has at least 2 peaks (in this case, the polymer has at least 2 Tgs), the Tg of the test polymer is the temperature at which the graph has the greatest amplitude peak (i.e. corresponding to the largest value of TANδ; in this case only the “major” Tg is considered to be that for the test polymer).

In the present invention, the transition temperature Tg1 corresponds to the “major” Tg (as defined above) of the first film-forming polymer when it has at least 2 Tgs; the glass transition temperature Tg2 corresponds to the “major” Tg of the second film-forming polymer when it has at least 2 Tgs.

The first film-forming polymer and the second film-forming polymer may be selected independently of each other from radical type polymers, polycondensates and polymers of natural origin as defined above having the glass transition temperature characteristics defined above.

First film-forming polymers in aqueous dispersion which may be used are aqueous dispersions of polymers sold under the trade names “NeoRez R-989®” by AVECIA RESINS, “Avalure® UR-405” by NOVEON or “Bayderm Finish DLH®” by BAYER.

By way of example, second film-forming polymers in aqueous dispersion which may be used are aqueous dispersions of polymers sold under the trade name “Avalure® UR-460” by NOVEON or “Acrilem IC89RT®” by ICAP.

The film-forming polymer of the aqueous dispersion “Avalure UR-460” is a polyurethane obtained by polycondensation of tetramethylene polyoxide, tetramethylxylylene diisocyanate, isophorone diisocyanate, and dimethylolpropionic acid.

In a particular implementation of this third variation of the invention, the aqueous dispersion of particles is an aqueous dispersion of particles of at least one polyurethane and at least one radical type polymer with a carboxylic group.

An aqueous dispersion comprising particles of polyurethane and radical type polymer with a carboxylic group in accordance with the invention may, for example, be prepared by simply mixing an aqueous polyurethane dispersion and an aqueous dispersion of radical type polymer, or by directly forming a dispersion of a mixture of particles of polyurethane and radical type polymer.

The aqueous polyurethane dispersion may, for example, be an aqueous dispersion of anionic polyurethane, polyester-polyurethane, and/or polyether-polyurethane, used alone or as a mixture, which may have a dry matter content of 10-50%.

As an example, in this variation the present invention may use a dispersion of polyurethane and a dispersion of radical type polymers obtained by mixing the following, then stirring for about 30 minutes at ambient temperature under the following conditions:

(i) polymers employed:

| | |
|---|---|
| radical, Tg = 35° C. | “Joncryl 77 ®” acrylic |
| (termed polymer 1A): | styrene dispersion from |
| | Johnson, 46% dry matter |
| radical, Tg = 65° C. | “Neocryl XK 63 ®” acrylic |
| (termed polymer 1B): | styrene dispersion from |
| | ICI, 44% dry matter |
| radical, Tg = 110° C. | “Joncryl 90 ®” acrylic |
| (termed polymer 1C): | styrene dispersion from |
| | Johnson, 44% dry matter |
| polyurethane, Tg = −10° C. | Dispersion of aliphatic |
| (termed polymer 2): | anionic polyurethane |
| | “IW/019.1 ®” from UCB, 35% |
| | dry matter |

(ii) proportion and nature of ingredients of mixture:

Polymer 1

| Polymer 2 % | | % | Dry extract % |
|---|---|---|---|
| 50 | A | 50 | 35 |
| 50 | B | 50 | 35 |
| 50 | C | 50 | 35 |

dry extract: amount of dry matter in the dispersion before evaporating;
%: % of polyurethane dispersion or radical type dispersion in the mixture; i.e. before evaporating.

The particles of the first and second film-forming polymers may have a mean size, independently of each other, of 10 nm to 500 nm, especially 20 nm to 300 nm.

In the dispersion used in the third variation of the invention, the film-forming polymer is generally present in an amount of 50% to 90% by weight relative to the total weight of the first and second polymers. By analogy, the second film-forming polymer is thus present in the dispersion in an amount of 10% to 50% by weight relative to the total weight of the first and second film-forming polymers.

In a particular implementation, the first film-forming polymer may be present in the mixture of polymers in an amount of 70% to 90% by weight relative to the total weight of the first and second film-forming polymers, more particularly 70% to 85% by weight. The second polymer is then present in the mixture of film-forming polymers in an amount of 10% to 30% by weight respectively relative to the total weight of the first and second film-forming polymers, more particularly 15% to 30% by weight.

In this third variation, the total weight of particles of the first film-forming polymer and the second film-forming polymer is generally 0.1% to 60% by weight relative to the total weight of the dispersion, in particular 1% to 50% and more particularly 5% to 40% by weight.

In a fourth variation of the invention, the aqueous dispersion used comprises at least one particular multi-phased aqueous dispersion.

More precisely, it comprises a dispersion of particles in an acceptable aqueous medium, the particles comprising at least one flexible phase at least the outer portion of which comprises at least one flexible polymer having at least one glass transition temperature of 60° C. or less and at least one rigid phase in at least the inner portion, the rigid phase being an amorphous material having at least one glass transition temperature of more than 60° C., the flexible polymer being at least partially fixed onto the rigid phase by chemical grafting.

The particles of the invention, also termed multiphased (or composites), are particles comprising at least one flexible phase and at least one rigid phase.

The flexible polymer of the particles in dispersion has at least one glass transition temperature of 60° C. or less, especially from −120° C. to 60° C., in particular 45° C. or less, especially from −120° C. to 45° C., and more particularly 30° C. or less, especially from −120° C. to 30° C.

The flexible polymer may be selected from block and/or random polymers. The term “block and/or random type polymers” means polymers in which the distribution of monomers on the principal chain or the pendant chains is in blocks and/or random.

The flexible polymer may be selected from radical type polymers, polycondensates, and silicone polymers. The flexible polymer may be selected from polyacrylics, polymethacrylics, polyamides, polyurethanes, polyolefins especially polyisoprenes, polybutadienes, polyisobutylenes (PIB), polyesters, polyvinylethers, polyvinylthioethers, polyoxides, polysiloxanes and especially polydimethylsiloxanes (PDMS), and combinations thereof. The term “combinations” means copolymers which may be formed from monomers leading to the formation of said polymers.

In particular, the flexible polymer may be selected from poly(meth)acrylics, polyurethanes, polyolefins and polysiloxanes.

The amorphous material of the rigid phase has a glass transition temperature of more than 60° C., especially more than 60° C. and 200° C. or less, in particular 70° C. or more, especially from 70° C. to 200° C., more particularly from 70° C. to 150° C., or even 90° C. or more, especially 90° C. to 150° C.

The amorphous material of the rigid phase may be a polymer, in particular a block and/or random type polymer. It may be a polymer selected from polyacrylics, polymethacrylics such as poly (meth)acrylic acid, poly(meth)acrylamides, polyvinyls, polyvinylesters, polyolefins, polystyrenes, polyvinylhalides such as polyvinyl chloride (PVC), polyvinylnitriles, polyurethanes, polyesters, polyamides, polycarbonates, polysulfones, polysulfonamides, polycyclics having a carbonaceous ring in the principal chain, such as polyphenylenes, polyoxyphenylenes, and combinations thereof.

Advantageously, the amorphous material of the rigid phase may be a polymer selected from polyacrylics, polymethacrylics such as poly (meth)acrylic acid, for example, poly(meth)acrylamides, polyvinyls, polyvinylesters, polyolefins, polystyrenes, polyvinylhalides such as polyvinyl chloride (PVC), polyvinylnitriles, polyurethanes, polyamides, and polyesters.

In a particular implementation of the invention, the flexible and rigid phases of the multiphase particles may comprise at least one radical type polymer obtained by or essentially by polymerizing monomers selected from the group formed by:

esters of (meth)acrylic acid such as alkyl (meth)acrylates, especially containing a C₁-C₈ alkyl group;

vinyl esters of linear or branched carboxylic acids, such as vinyl acetate or vinyl stearate;

styrene and its derivatives, such as chloromethyl styrene or alpha methyl styrene;

conjugated dienes, such as butadiene or isoprene;

acrylamide, methacrylamide, and acrylonitrile;

vinyl chloride; and

(meth)acrylic acid.

Selection of the monomers (nature and amount), which monomers may be a single monomer or a mixture of at least two monomers, for the flexible polymer and the amorphous material of the rigid phase, is determined by the glass transition temperature with which each polymer is to be endowed.

The polymers of the rigid and/or flexible phase may be cross-linked using monomers having at least two copolymerizable double bonds, for example selected from:

conjugated dienes, such as butadiene or isoprene;

allyl esters of alpha, beta-unsaturated carboxylic acids, such as allyl acrylate, allyl methacrylate;

allyl esters of dibasic alpha, beta-unsaturated carboxylic acids, such as diallyl maleate;

polyacrylics or polymethacrylics generally comprising at least two ethylenically unsaturated bonds, such as ethylene glycol dimethacrylate, 1,3-butylene glycol dimethacrylate, 1,4-butanediol diacrylate or pentaneerythritol tetraacrylate;

polyvinyl compounds, such as divinylbenzene or trivinylbenzene; and

polyallyl compounds, such as triallyl cyanurate.

By forming covalent bonds, chemical grafting can bond the rigid phase and the flexible phase of the multi phase particles in a stable manner.

Chemical grafting may be carried out by sequenced radical type polymerization (also termed block polymerization) using operating modes which are well known to the skilled person. Block polymerization consists of polymerizing, in a first step, the monomers of the rigid polymer (polymer forming the rigid phase of the particles) then continuing during a second polymerization step with monomers forming the flexible polymer (polymer forming the flexible phase of the particles). In this manner, the polymer chains of the flexible phase are at least partially bonded by covalent bonding to the chains of the polymer of the rigid phase, the covalent bonding resulting from polymerizing a monomer of the flexible polymer with a monomer of the rigid polymer. Advantageously, the monomers of the polymer of the outer flexible phase have a greater affinity for the dispersion medium than the monomers of the polymer of the inner rigid phase.

The flexible polymer may be grafted onto the rigid polymer via the grafting monomer, which may possibly be a monomer having a plurality of double bonds (ethylenically unsaturated bonds), in particular a monomer having two ethylenically unsaturated double bonds. The grafting monomer may be a conjugated diene such as those described above, or an allyl ester (in particular a diester) of alpha, beta-unsaturated dicarboxylic acids such as those described above (such as diallyl maleate) which have two polymerizable functions (ethylenically unsaturated double bond) with different reactivities: one of the polymerizable functions (ethylenically unsaturated double bond) of the grafting monomer is polymerized with the polymer of the amorphous material of the rigid phase (rigid polymer), and the other polymerizable function (ethylenically unsaturated double bond) of the same grafting monomer is polymerized with the flexible polymer.

When the flexible polymer or the polymer of the rigid phase is a polycondensate, a polycondensate is in particular used which has at least one ethylenically unsaturated bond which is capable of reacting with a monomer also comprising an ethylenically unsaturated bond to form a covalent bond with the polycondensate. Polycondensates comprising one or more ethylenically unsaturated bonds are obtained by polycondensing monomers such as allyl alcohol, vinylamine or fumaric acid. As an example, it is possible to polymerize vinyl monomers with a polyurethane containing vinyl groups in or at the end of the polyurethane chain and thus to graft a vinyl polymer onto a polyurethane; a dispersion of particles of such a graft polymer is described in the publications “The structure and properties of acrylic-polyurethane hybrid emulsions”, M. Hiroze, Progress in Organic Coatings, 38 (200), pages 27-34; “Survey of the applications, properties, and technology of cross-linking emulsions”, B. Bufkin, Journal of Coatings Technology, vol 50, no 647, December 1978.

The same grafting principle is applicable to silicones using silicones comprising vinyl groups, allowing polymerization of vinyl monomers onto the silicone, thus allowing vinyl polymer chains to be grafted onto a silicone.

The particles of the rigid and flexible phases generally have a size of 1 nm to 10 μm, in particular 10 nm to 1 μm.

The flexible phase may be present in the particles in an amount of at least 1% by volume relative to the total particle volume, especially at least 5% by volume, in particular at least 10% by volume, and more particularly at least 25% by volume. The flexible phase may be present in the particles in an amount of up to 99.999% by volume, especially up to 99.9% by volume, in particular up to 99% by volume, and more particularly up to 95% by volume. In particular, the flexible phase may be present in the particles in an amount of 1% to 99.999% by volume, in particular 5% to 99.9% by volume and more particularly 10% to 99.9% by volume, especially 25% to 99.9% by volume and more particularly 50% to 95% by volume or even 50% to 99% by volume.

In all cases, the rigid phase and the flexible phase are incompatible, i.e. they may be distinguished using techniques which are well known to the skilled person, such as observation using an electron microscope or measuring the glass transition temperatures of the particles by differential calorimetry. The multiphase particles are thus non-homogeneous particles.

The morphology of the flexible and rigid phases of the dispersed particles may, for example, be of the core-shell type with the shell parts completely surrounding the core, but also of the core-shell type with a multiplicity of cores or an interpenetrating network of phases. In the multiphase particles, the flexible phase is at least partially, in particular mainly external and the rigid phase is at least partially, in particular mainly internal.

The multiphase particles may be prepared using series of consecutive polymerization steps with different types of monomers. The particles of a first category of monomers are generally prepared in a separate step or formed in situ by polymerization. Next or at the same time, at least one other category of other monomers is polymerized during at least one additional polymerization step. The particles formed have at least one at least partially internal structure, or core, and at least one structure which is at least partially external, or the shell. Forming a heterogeneous “multi-layered” structure is thus possible. A wide variety of morphologies of the core-shell type may flow from this, but fragmented inclusions of the rigid phase in the flexible phase, for example, are also possible. According to the invention, it is essential for the structure of the at least partially external flexible phase to be more flexible than the at least partially internal rigid phase structure.

In a particular implementation of the invention, the multiphase particles may be dispersed in an aqueous medium, in particular a hydrophilic medium. The aqueous medium may be mainly constituted by water, in particular practically completely constituted by water. Said dispersed particles thus form an aqueous dispersion of polymer, generally known as a latex or pseudo-latex. The term “latex” means an aqueous dispersion of polymer particles which may be obtained by emulsion polymerization of at least one monomer.

The dispersion of multiphase particles is generally prepared by at least one emulsion polymerization step, in an essentially aqueous continuous phase, starting from reaction initiators such as photochemical or thermal initiators for a radical type polymerization, optionally in the presence of additives such as stabilizers, chain transfer agents and/or catalysts.

Aqueous dispersions of film-forming polymers which may be used are acrylic dispersions sold under the trade names “Neocryl XK-90®”, “Neocryl A-1070®”, “Neocryl A-1090®”, “Neocryl BT-62®”, “Neocryl A-1079®” and “Neocryl A-523®” by AVECIA-NEORESINS, “Dow Latex 432®” by DOW CHEMICAL, “Daitosol 5000 AD®” or “Daitosol 5000 SJ” by DAITO KASEY KOGYO; “Syntran 5760” by Interpolymer or aqueous dispersions of polyurethane sold under the trade names “Neorez R-981®” and “Neorez R-974®” by AVECIA-NEORESINS, “Avalure UR-405®”, “Avalure UR-410®”, “Avalure UR-425”, “Avalure UR-450®”, “Sancure 875®”, “Sancure 861®”, “Sancure 878®” and “Sancure 2060®” by GOODRICH, “Impranil 85®” by BAYER, “Aquamere H-1511®” by HYDROMER sulfopolyesters sold under the registered trade mark “Eastman AQ®” by EASTMAN CHEMICAL PRODUCTS, vinyl dispersions such as “Mexomere PAM”, aqueous polyvinyl acetate dispersions such as “Vinybran®” from Nisshin Chemical or those sold by UNION CARBIDE, aqueous dispersions of terpolymers of vinyl pyrrolidone, dimethylaminopropyl methacrylamide and lauryldimethylpropylmethacrylamidoammonium chloride such as “Styleze W®” from ISP, aqueous dispersions of hybrid polyurethane/polyacrylic polymers , such as those sold under references “Hybridur®” by AIR PRODUCTS or “Duromer®” by NATIONAL STARCH, dispersions of particles of the core-shell type such as those sold by ATOFINA with reference “Kynar®” (core: fluorine-containing; shell acrylic) or those described in U.S. Pat. No. 5,188,899 (core silica; shell: silicone) and mixtures thereof.

The aqueous dispersions of particles used in the invention may also comprise different additives. They may comprise an auxiliary film-forming agent encouraging the formation of a film with particles of film-forming polymers. An auxiliary film-forming agent may be selected from any of the compounds known to the skilled person to be capable of fulfilling the desired function and in particular may be selected from plasticizing agents and coalescing agents.

### Plasticizer

In a particular implementation, the dispersion used in the invention comprises an agent termed a plasticizer. This agent, which can plasticize the polymer in aqueous dispersion, is generally an organic compound having a distribution coefficient D of 0.1 or less. The distribution coefficient is determined as disclosed in the document published in the review “Progress in Organic Coatings”, vol 30, 1997, pp 173-177 entitled “A method to predict the distribution coefficient of coalescing agents between latex particles and the water phase”.

The plasticizing agent may in particular be selected from diisobutyl adipate, the ester of tertio-butyl acid and 2,2,4-trimethylpentane-1,3-diol, diethyl adipate, diethyl phthalate, dibutyl phthalate, diocyl phthalate, butyl phthalate and 2-ethylhexyl phthalate, dimethyl sebacate, dibutyl sebacate, ethyl stearate, 2-ethylhexyl palmitate, dipropylene glycol n-butyl ether and mixtures thereof.

Advantageously, the plasticizing agent is selected from diisobutyl adipate, the ester of tertio-butyl acid and 2,2,4-trimethylpentane-1,3-diol, dipropylene glycol n-butyl ether and mixtures thereof.

The plasticizing agent may be present in the dispersion used in the invention in an amount of 0.1% to 20% by weight, in particular 0.5% to 10% relative to the total dispersion weight.

When the aqueous dispersion of particles is obtained using the third variation of the invention, i.e. when the aqueous dispersion used comprises a mixture of at least two film-forming polymers in the form of solid particles which are distinguished by their respective glass transition temperatures Tg (Tg1 being 30° C. or more and Tg2 being 0° C. or less), the dispersion may advantageously be free or substantially free of plasticizing agent.

### Coalescing Agent

In a particular implementation, the dispersion used in the invention may also comprise a coalescing agent. This agent, which encourages coalescence of polymer particles in aqueous dispersion, is generally an organic solvent having a distribution coefficient Dt of 0.5 or more, as disclosed in the document published in the review “Progress in Organic Coatings”, vol 30, 1997, pp 173-177 entitled “A method to predict the distribution coefficient of coalescing agents between latex particles and the water phase”.

The coalescing agent which may be used in the invention may be propylene glycol n-butyl ether, dipropylene glycol dimethyl ether, propylene glycol methyl acetate ether, propylene glycol propyl ether, methyl lactate, ethyl lactate, isopropyl lactate, or mixtures thereof.

Advantageously, the coalescing agent is selected from propylene glycol n-butyl ether, dipropylene glycol dimethyl ether, isopropyl lactate, and mixtures thereof.

The coalescing agent may be present in the dispersion in an amount of 0.1% to 15% by weight, in particular 0.5% to 8% by weight relative to the total dispersion weight.

In a particular implementation, the film of the present invention may be obtained by drying the aqueous dispersion or mixture of aqueous dispersions of particles of polymers by heating, for example to a temperature of 40° C. to 150° C. In such a case, the dispersion may advantageously be free of coalescing agent.

### Additional Particulate Phase

The aqueous dispersion used to form the film may also comprise an additional particulate phase in an amount of 0.01% to 30%, especially 0.01% to 15%, in particular 0.02% to 10% and more preferably 0.05% to 10% by weight relative to the total weight of the corresponding article. It may be at least one coloring substance, especially a pigment and/or at least one nacre and/or at least flakes and/or at least one complementary filler used in cosmetic compositions.

The term “pigments” means white or colored particles, which may be mineral or organic, intended to color and/or opacify the film resulting from the dispersion. The term “complementary fillers means colorless or white particles, which may be mineral or synthesized, lamellar or non lamellar. The term “nacres” means iridescent particles, in particular produced in the shells of certain mollusks or synthesized. These fillers and nacres serve to modify the texture of the film resulting from the dispersion.

The pigments may be present in an amount of 0.01% to 15% by weight, in particular 0.01% to 10% by weight, more particularly 0.02% to 5% by weight. Mineral pigments which are suitable in the context of the invention which may be mentioned are oxides of titanium, zirconium or cerium and oxides of zinc, iron or chromium, ferric blue, manganese violet, ultramarine blue and chromium hydrate.

Organic pigments which may be used in the context of the invention which may be mentioned are carbon black, D&C type pigments, lakes based on cochineal carmine, barium, strontium, calcium or aluminium, or the diketo pyrrolopyrroles (DPP) described in EP-A-0 542 669, EP-A-0 787 730, EP-A-0 787 731 and WO-A-96/08537.

The nacres may be present in the resulting film in an amount of 0.01% to 20% by weight, preferably 0.01% to 15% by weight, and more preferably 0.02% to 10% by weight relative to the total weight of the resulting film. The nacre pigments may be selected from white nacre pigments such as mica coated with titanium or bismuth oxychloride, colored nacre pigments such as mica titanium with iron oxides, mica titanium with ferric blue or chromium oxide, mica titanium with an organic pigment of the type mentioned above, as well as nacre pigments based on bismuth oxychloride.

The complementary fillers may be present in an amount of 0.01% to 20% by weight, preferably 0.01% to 15% by weight and more preferably 0.02% to 10% by weight relative to the total weight of the article.

In particular, the following may be mentioned: talc, zinc stearate, mica, kaolin, polyamide powders (Nylon®) (Orgasol® from Atochem), polyethylene powders, tetrafluoroethylene polymer powders (Teflon®), starch, boron nitride, polymer microspheres such as those formed from polyvinylidene chloride/acrylonitrile such as Expancel® (Nobel Industrie), acrylic acid copolymers (Polytrap® from Dow Corning), silicone resin microbeads (Tospearls® from Toshiba, for example) and organopolysiloxane elastomers.

The dispersion may also comprise hydrosoluble or liposoluble colorants in an amount of 0.01% to 10% by weight, especially 0.01% to 5% by weight relative to the total weight of the resulting film. Examples of liposoluble colorants are Sudan red, DC Red 17, DC Green 6, β-carotene, soya oil, Sudan brown, DC Yellow 11, DC Violet 2, DC orange 5 and quinoline yellow. Examples of hydrosoluble colorants are beetroot juice and methylene blue.

The dispersion of the invention may also contain ingredients which are routinely used in cosmetics and more particularly in the cosmetics and/or nail care fields. They may in particular be selected from vitamins, oligo-elements, softeners, sequestrating agents, alkalinizing or acidifying agents, spreading agents, wetting agents, thickening agents, dispersing agents, anti-foaming agents, preservatives, UV filters, active ingredients, moisturizing agents, fragrances, neutralizing agents, stabilizing agents, antioxidants and mixtures thereof.

Thus, when the dispersions of the invention are more particularly intended for the care of natural nails, they may incorporate, as active ingredients agents, for hardening keratinous substances, active ingredients acting on the growth of the nail, for example methyl sulfonyl methane, and/or active ingredients to treat various diseases of the nail such as onichomycosis.

The quantities of these various ingredients are those conventionally used in this field, for example 0.01% to 20% and in particular 0.01% to 10% by weight relative to the total weight of the article.

In a particular implementation of the invention, the aqueous dispersion of polymers may advantageously be partially neutralized, which limits the quantity of ionized functions. This is possible because the aqueous dispersion does not have to be stable within the context of the present invention. This implementation results in an article which is advantageously more resistant to water.

In a particular implementation of the invention, the aqueous dispersion used may be free of laponite gel or aqueous gelling agent normally used to stabilize pigments in aqueous dispersions. An article of the invention produced with such a dispersion has increased water resistance.

### Adhesive Material

The article of the invention has an adhesive outer face. Said adhesive face is generally obtained by dint of the presence of at least one layer of at least one adhesive material.

The term “material” as used in the context of the present invention means a polymer or a polymeric system which may comprise one or more polymers of different natures. Said adhesive material may be in the form of a solution of polymer or a dispersion of polymer particles in a solvent. Said adhesive material may also contain a plasticizer as defined above. Said adhesive material may have a certain adhesive power as defined by its viscoelastic properties.

The viscoelastic properties of a material are conventionally defined by two characteristic values which are as follows:

the elastic modulus, which represents the elastic behavior of the material for a given frequency and which is conventionally denoted G′;

the viscous modulus, which represents the viscous behavior of the material for a given frequency which is conventionally denoted G”.

These magnitudes have been defined in the “Handbook of Pressure Sensitive Adhesive Technology” 3^{rd} edition, D. Satas, chap. 9, p. 155 to 157.

Adhesive materials which can be used in the context of the present invention have viscoelastic properties which are measured at a reference temperature of 35° C. and in a certain frequency range.

In the case of adhesive materials in the form of a solution or dispersion of polymer in a volatile solvent (such as water, a short chain ester, a short chain alcohol, acetone, etc), the viscoelastic properties of the material are measured under conditions under which it has a volatile solvent content of less than 30%, in particular a volatile solvent content of less than 20%.

In particular, the elastic modulus of the material is measured at three different frequencies:

at low frequency, i.e. 2×10⁻² Hz;

at an intermediate frequency, i.e. 0.2 Hz;

at high frequency, i.e. at 2 Hz;

and the viscous modulus at the frequency of 0.2 Hz.

These measurements allow the change of adhesive power of the adhesive material over time to be measured.

These viscoelastic properties are measured during dynamic tests under low amplitude sinusoidal stresses (small deformations) carried out at 35° C. over a frequency range of 2×10⁻² to 20 Hz using a “Haake RS50” type rheometer under tension/shear stress, for example in cone/plane geometry (for example with a cone angle of 1°).

Advantageously, said adhesive material satisfies the following conditions:

G′ (2 Hz, 35° C.)≧10³ Pa; and

G′ (35° C.)≦10⁸ Pa, in particular G (35° C.)≦10⁷ Pa;

G′ (2×10⁻² Hz, 35° C.)≦3.10⁵ Pa; in which:
G′ (2 Hz, 35° C.) is the elastic shear modulus of said adhesive material, measured at a frequency of 2 Hz and at a temperature of 35° C.;
G′ (35° C.) is the elastic shear modulus of said adhesive material measured at a temperature of 35° C., for any frequency in the range 2×10⁻² to 2 Hz;
G′(2×10⁻² Hz, 35° C.) is the elastic shear modulus of said adhesive material, measured at a frequency of 2×10⁻² Hz and at a temperature of 35° C.

In a particular form of the invention, the adhesive material also satisfies the following condition:

G″/G′ (0.2 Hz, 35° C.)≧0.35. in which:
G″ (0.2 Hz, 35° C.) is the viscous shear modulus of said adhesive material, measured at a frequency of 0.2 Hz and at a temperature of 35° C.;
G′ (0.2 Hz, 35° C.) is the elastic shear modulus of said adhesive material, measured at a frequency of 0.2 Hz and at a temperature of 35° C.

In a particular form of the invention, we have

G′ (2 Hz, 35° C.)≧5×10³ Pa, and in particular, G′ (2 Hz, 35° C.)≧10⁴ Pa.

In a further particular form of the invention, we have:

G′(2×10⁻² Hz, 35° C.)≦5×10⁴ Pa.

In particular, the adhesive materials of the invention satisfy the following four conditions:

G′ (2 Hz, 35° C.≧10⁴ Pa; and

G′ (35° C.)≦10⁸ Pa, in particular G′(35° C.)≦10⁷ Pa;

G′(2×10⁻² Hz, 35° C.)≦5×10⁴ Pa; and

G″/G′(0.2 Hz, 35° C.)≧0.35.

The adhesive materials of the invention may be selected from adhesives of the “Pressure sensitive adhesive” type, for example those mentioned in the “Handbook of pressure sensitive adhesive technology”, 3^{rd} edition, D Satas.

In general, the adhesive layer is such that said article cannot be removed by peeling when it is applied to the surface of a synthetic or natural nail after leaving for at least 24 hours.

The adhesive materials of the invention are polymers selected from block or random copolymers comprising at least one monomer or an association of monomers, the resulting polymer having a low glass transition temperature at ambient temperature (25° C.), said monomers or associations of polymers possibly being selected from butadiene, ethylene, propylene, isoprene, isbutylene, a silicone, and mixtures thereof. Examples of such materials are styrene-butadiene-styrene, styrene-(ethylene-butylene)-styrene, styrene-isoprene-styrene type block copolymers such as those sold under the trade name “Kraton®” by SHELL CHEMICAL Co. or “Vector®” from EXXON.

The adhesive materials of the invention are in particular adhesive polymers selected from:

polyurethanes;

acrylic polymers;

silicones;

butyl gums, in particular polyisobutylenes;

ethylene-vinyl acetate polymers;

polyamides, optionally modified by fatty chains;

natural gums; and

mixtures thereof.

In particular, they may be adhesive copolymers deriving from copolymerizing vinyl monomers with polymeric entities such as those described in U.S. Pat. No. 6,136,296, for example. The invention also encompasses the adhesive copolymers described in U.S. Pat. No. 5,929,173 having a polymeric skeleton, with a Tg of 0° C. to 45° C., grafted by chains deriving from acrylic and/or methacrylic monomers and, in contrast, having a Tg of 50° C. to 200° C.

The adhesive materials are, for example, selected from polyisobutylenes having a relative molar mass Mv of 10000 or more to 150000 or less. In particular, said relative molar mass is 18000 or more to 150000 or less.

Commercially available products which are of particular service in the present invention which may be mentioned are polyisobutylenes with a relative molar mass Mv of 40000, 55000 and 85000 sold respectively under the trade names “Oppanol B 10®”, “Oppanol B 12®” and “Oppanol B 15®” by BASF, and mixtures thereof.

The adhesive material in the article of the invention is generally in the form of a layer with a thickness of 1 μm to 100 μm and in particular from 1 μm to 50 μm. preferably 1 μm to 25 μm.

In a particular implementation of the invention, the layer formed by the adhesive material is in direct contact with the polymeric film, obtained by evaporating off the aqueous phase of the aqueous dispersion of particles of at least one film-forming polymer.

Advantageously, the adhesive material and the film are compatible because of their chemical nature and composition. In one particular implementation, the solvent for the adhesive can result in an increase in the mass of the film in contact therewith, in particular by at least 10% by weight relative to the initial weight of the film. In other words, this increase results in the film increasing in mass.

In a variation of the invention, the article has an intermediate layer between the layer of adhesive material and the film obtained by evaporating the solvent phase, which may be organic or aqueous, of a solution or dispersion of at least one film-forming polymer, which may or may not be colored. Said layer may in particular be constituted by a film of polish in a solvent phase based on nitrocellulose and/or at least a cellulose ester, in particular colored. Such an architecture is particularly advantageous as regards staying power. The polymeric film effectively protects the film of polish against shocks and thus significantly extends its staying power.

The article of the present invention may be in various forms, such as a star, square, circle, etc.

As described above, the present invention also encompasses a product suitable for packaging an article of the present invention in the partially dry form.

Once applied, the article of the present invention is dried and then adopts its definitive structure by contact with ambient air.

The article of the invention generally has a thickness of 1 μm to 500 μm, especially 1 μm to 300 μm and in particular 1 μm to 200 μm.

As mentioned above, the article of the invention is covered at least on its outer adhesive face with a removable support.

Said support may be of any nature compatible with the fact that while it is in contact with an adhesive material, it may nevertheless be separated therefrom.

The removable support defined above may be in the form of a protective layer consisting, for example of a film, in particular a plastic film or paper or a sheet type textile structure.

Advantageously, said support is constituted by a transparent material to prevent any error in the choice of color. It may be constituted by one or more layers which may have different natures. As an example, it may be a sheet of paper covered with one of the plastics mentioned below.

Examples of suitable plastic films which may be used in the article of the invention which may be mentioned are films formed from polyesters, for example polyethylene terephthalates, polybutylene terephthalates or polyethylene sebacates or made from polyethylene, polypropylene or polyamides such as polyhexamethylene adipate, polycaprolactame or poly(omega-ω-undecanoic acid amide). Because of its surface characteristics, these plastics are clearly not removable per se. To provide this characteristic, it is necessary to carry out a surface treatment using appropriate substances, such as a treatment with silicones or, particularly advantageously, a treatment with salts of long chain fatty acids such as C₁₂-C₂₂, for example, said acids being saturated or possibly containing up to three olefinic bonds, and at least divalent metals, in particular salts of heavy transition metals of this type and in particular chromium salts.

The textile sheet type structure may be woven or non-woven.

In a particular implementation, both faces of the article of the invention are covered with a removable support which may be identical or different.

As indicated above, the present invention also provides a method of preparing a flexible article for making up and/or for caring for the nails. An article of the invention may in particular be obtained with a device as described in U.S. Pat. No. 4,903,840.

The method of the invention comprises a step of evaporating the aqueous dispersion of particles of film-forming polymers to obtain a film. Said evaporation may be achieved using conventional methods which are well known to the skilled person. It may be achieved by partial drying, in particular by heating, for example at a temperature of 20° C. to 150° C.

In a particular implementation of the method of the invention, the layer of the aqueous dispersion of particles of film-forming polymer has a thickness which may measure from 1 μm to 300 μm, in particular 1 μm to 150 μm.

The adhesive material is generally deposited in the form of a layer of material with a thickness of 0.5 μm to 200 μm, in particular 1 μm to 100 μm.

This method may take its inspiration from the method described in U.S. Pat. No. 5,415,903.

The article obtained, in particular the excess film, is then generally cut, before or after application, to the desired size and form with small scissors, with nail-clippers, or by scratching the film.

The present invention also provides a method of making up the nails, in which the article as defined above is applied.

The effect obtained may be eliminated using makeup removers which are in routine use in the nail polish field.

1. A flexible article for making up and/or caring for the nails and/or false nails, comprising at least:
an adhesive layer; and
at least one polymeric film obtained by evaporating the aqueous phase from an aqueous dispersion of particles of at least one film-forming polymer.

2. An article according to claim 1, wherein it further comprises at least one film of colored polish between the adhesive layer and the polymeric film.

3. An article according to claim 1, wherein said polymeric film is transparent.

4. An article according to claim 2, wherein the film of polish is obtained by cross-linking or evaporating the organic or aqueous solvent phase from a solution or dispersion of at least one film-forming polymer.

5. An article according to claim 4, wherein the film is based on nitrocellulose and/or a cellulose ester.

6. An article according to claim 1, wherein it has a dry matter content of more than 80% by weight relative to its total weight.

7. An article according to claim 1, wherein it has a water take-up at 25° C. of 20% or less.

8. An article according to claim 1, wherein it has a storage modulus E′ of 1 MPa or more, at a temperature of 30° C. and a frequency of 0.1 Hz

9. An article according to claim 1, wherein it has a deformation at break εᵣ of 5% or more, and/or an energy at break per unit volume W of 0.2 J/cm3or more.

10. An article according to claim 1, wherein the size of the polymer particles of said aqueous dispersion is from 5 nm to 500 nm.

11. An article according to claim 1, that wherein said film-forming polymer is selected from synthetic radical type polymers, synthetic polycondensate type polymers and polymers of natural origin which may be modified.

12. An article according to claim 11, wherein said radical type synthetic polymer is a polymer of the homopolymer or copolymer type, which may be vinyl and/or acrylic.

13. An article according to claim 11, wherein said synthetic polycondensate type polymer is selected from anionic, cationic, non ionic or amphoteric polyurethanes, polyurethane-acrylics, polyurethane-polyvinylpyrrolidones, polyester-polyurethanes, polyether-polyurethanes, polyureas, polyurea/polyurethanes, silicone polyurethanes, polyesters, polyester amides, fatty chain polyesters, polyamides and epoxyester resins.

14. An article according to claim 11, wherein said polymer of natural origin, which may be modified, is selected from shellac resin, sandarac gum, dammars, elemis, copals, water-insoluble cellulose polymers such as nitrocellulose and modified cellulose esters.

15. An article according to claim 1, wherein said aqueous dispersion is an aqueous dispersion of polyester-polyurethane particles.

16. An article according to claim 1, wherein said aqueous dispersion of polymer particles is an aqueous dispersion of acrylic polymer which has solubility properties at 25° C. in organic solvents corresponding to the HANSEN solubility parameters dD, dP and dH satisfying the following conditions:
dD=17.5;
dP=7;
dH=7.6;
with a radius R of 5 to 10.

17. An article according to claim 1, wherein said dispersion is a dispersion comprising, in an acceptable aqueous medium:
a) solid particles dispersed in the aqueous medium of a first film-forming polymer having at least one glass transition temperature, Tg1, of 30° C. or more; and
b) solid particles dispersed in the aqueous medium of a second film-forming polymer having at least one glass transition temperature, Tg2, of 0° C. or less;
the first film-forming polymer being present in an amount of 50% to 90% by weight relative to the total weight of the first and second film-forming polymers.

18. An article according to claim 17, wherein said aqueous dispersion is an aqueous dispersion of particles of at least one polyurethane and at least one radical type polymer with a carboxylic group.

19. An article according to claim 1, wherein said dispersion is a dispersion of particles, the particles comprising at least one flexible phase which is at least partially external, comprising at least one flexible polymer having at least one glass transition temperature of 60° C. or less and at least one rigid phase which is at least partially internal, the rigid phase being an amorphous material having at least one glass transition temperature of more than 60° C., the flexible polymer being at least partially fixed by chemical grafting to the rigid phase.

20. (canceled)

21. An article according to claim 1, wherein said polymeric film is a multi-layered film produced by superposing at least two or more layers respectively obtained by evaporating the aqueous phase from dispersions of particles of film-forming polymers(s) of different natures and wherein said film is produced from:
a first aqueous dispersion of at least one film-forming polymer having at least one, glass transition temperature, Tg1, of 20° C. or less, said first dispersion being disposed on a support then dried at a temperature of 50° C. to 150° C.; and
a second aqueous dispersion deposited on the layer of the first dispersion, said second aqueous dispersion of at least one film-forming polymer having at least one, glass transition temperature, Tg2, of 30° C. or more;
the ensemble then being dried at a temperature of 50° C. to 150° C.

22. (canceled)

23. (canceled)

24. (canceled)

25. An article according to claim 1, wherein said adhesive layer comprises at least one adhesive material.

26. An article according to claim 1, wherein the adhesive layer is such that said article cannot be removed by peeling when it is applied to the surface of a synthetic or natural nail after at least 24 hours in position.

27. An article according to claim 25, wherein said adhesive material is selected from copolymers deriving from copolymerizing vinyl monomers with polymeric entities, copolymers having a polymeric skeleton, with a Tg from 0° C. to 45° C., grafted with chains deriving from acrylic and/or methacrylic monomers and having, in contrast, a Tg of 50° C. to 200° C., and polyisobutylenes having a relative molar mass Mv of 10000 or more to 150000 or less.

28. An article according to claim 1, wherein it further comprises a removable support, constituted by a plastic film modified by a surface treatment with silicone or with salts of C12 to C22 fatty acids.

29. A product for making up and/or caring for nails and/or false nails comprising, in a packaging which is substantially airtight, at least one article according claim 1, the packaging being such that said article is preserved in a partially dried form.

30. A product according to claim 29, wherein said article has a dry matter content of less than 80% by weight relative to the total weight of said article.

31. A product according to claim 29, wherein the packaging comprises a reservoir which can contain said article in a sealed manner.

32. A method of preparing a flexible article for making up and/or caring for the nails, comprising at least the steps consisting in superposing on a removable support:
a) at least one layer of a composition based on at least one adhesive material; and
b) at least one layer of an aqueous dispersion of particles of at least one film-forming polymer, the aqueous phase being evaporated off consecutive to deposition thereof to obtain a polymeric film.

33. A method according to claim 32, wherein furthermore, a film of colored varnish is formed between the adhesive layer and the polymeric film.

34. A method according to claim 33, wherein the film of polish is obtained by cross-linking and/or evaporating the organic or aqueous solvent phase from a solution or dispersion of at least one film-forming polymer.

35. (canceled)

36. (canceled)

37. A method of preparing a product according claim 29, comprising the following steps consisting in superposing on a removable support:
a) at least one layer of a composition based on at least one adhesive material;
b) at least one layer of an aqueous dispersion of particles of at least one film-forming polymer, the aqueous phase being evaporated from the film consecutive to depositing said composition and controlled to obtain a partially dry article; and
c) packaging said article in a partially dry state in substantially airtight packaging.

38. A method of making up the nails using an article as defined in claim 1, comprising applying the adhesive face of said article to a natural or synthetic nail.

39. An article according to claim 26, wherein said adhesive material is selected from copolymers deriving from copolymerizing vinyl monomers with polymeric entities, copolymers having a polymeric skeleton, with a Tg from 0° C. to 45° C., grafted with chains deriving from acrylic and/or methacrylic monomers and having, in contrast, a Tg of 50° C. to 200° C., and polyisobutylenes having a relative molar mass Mv of 10000 or more to 150000 or less.

40. A product according to claim 30, wherein the packaging comprises a reservoir which can contain said article in a sealed manner.
